(19) United States
(12) Patent Application Publication
(10) Pub. No.: US 2004/0013618 A1
(43) Pub. Date: Jan. 22, 2004

(54) Cosmetic formulation

(76) Inventor: Siro Passi, Rome (IT)

Correspondence Address: YOUNG & THOMPSON, 745 SOUTH 23RD STREET 2ND FLOOR, ARLINGTON, VA, 22202

(21) Appl. No.: 10/381,413

(22) PCT Filed: Sep. 27, 2001
(86) PCT No.: PCT/IT01/00495

(30) Foreign Application Priority Data
Sep. 28, 2000 (IT) RM2000A000520

(51) Int. Cl. A61K 7/42; A61K 7/06; A61K 31/704

(52) U.S. Cl. 424/59; 424/74; 514/33

(57) ABSTRACT

A cosmetic formulation, including active principles of strictly natural origin to protect skin against exogenous or endogenous harmful agents as well as to balance again the dermal homeostasis lipids altered by dermatosis and ageing, is characterized by a lipid composition which is close to the human sebum, has a pool of antioxidants similar to those present in sebum and skin, and has anti-inflammatory and anti-free-radical molecules capable of preventing histidine from being decarboxylated and then histamine to be formed.

The present invention relates to cosmetics and more particularly cosmetic formulations for topical administering to protect skin against exogenous or endogenous harmful agents as well as to balance again the dermal homeostasis lipids altered by dermatosis and ageing. Such formulations include active principles of strictly natural origin, all of them fulfilling the following requirements:

a) having a lipid composition which is close to the human sebum;

b) having a pool of antioxidants similar to those present in sebum and skin;

c) having anti-inflammatory and anti-free-radical molecules also capable of preventing histidine from being decarboxylated and then histamine to be formed. The rational principle of the present invention is to formulate and to prepare topical products of strictly natural origin which are capable of reproducing the human sebum so as to carry the active principles contained therein without the skin gives rise to self-defence systems against exogenous products by the contact with the carrier

Another object of the invention is to provide anallergenic cosmetic formulations which are unable to induce dermal allergic contact reactions.

In order to achieve such result, according to the invention, it is necessary to provide formulations containing active principles of strictly natural origin which fulfil three main requirements:

a) having a lipid composition which is as close as possible to the human sebum;

b) having a pool of antioxidants similar to those present in sebum and skin;

c) having anti-inflammatory and anti-free-radical molecules also capable of preventing histidine from being decarboxylated and then histamine to be formed.

The whole knowledge about chemistry and biology of dermal lipids brought to formulate a mixture of lipids similar to sebum of strictly natural origin in a previous Patent application of the same Applicant which is integral with the present description. Such formulation is close to the lipid composition of human sebum produced by oil glands.

A preferred formulation of such mixture (which will be referred to thereafter as oil A*) is as follows:

| | % |
|---|---|
| Macadamia oil | 50 |
| Jojoba oil (wax) | 24.5 |
| Avocado oil | 10 |
| Unsaponifiable olive oil (squalene) | 12 |
| Free cholesterol | 1.5 |
| Oily cholesterol | 0.5 |
| Soya phospholipids | 1.4 |
| BHT (butylhydroxytoluene) | 0.1 |

Therefore, a preparation having such formulation can provide a lipid component which is quite similar to the component present in the human sebum both from the quantitative and qualitative points of view according to above item a).

As far as item b) is concerned, i.e. the presence of antioxidants similar to those contained in human sebum and skin, the inventor has found recently, according to the researches carried out, that human sebum contains two main antioxidants, i.e. vitamin E (d-RRR-α-tocopherol) and ubiquinone (CoQ₁₀), which operate in syntony and synergy to protect sebum and particularly the polyunsaturated hydrocarbon squalene from the oxidation induced by solar radiations and other oxidizing agents present in the environment.

Therefore, vitamin E (0.5-1.5%) and ubiquinone (0.02-0.2%) together with magnesium ascorbyl phosphate or ascorbyl dipalmitate, a steady form of vitamin C (1-3%) the hydrolysis of which generates vitamin C, can be used as main natural physiological antioxidants in the formulations according to the present invention. In some cases, extremely active flavonoids such as rutin and those present in association with ginkgoales in ginkgo biloba can also be used.

The further characterizing component of the formulations of the present patent application is the presence of anti-inflammatory and anti-free-radical agents as well as inhibitors of the histidine decarboxylation.

It is known that histamine is formed from the decarboxylation of histidine, a molecule directly involved in the allergic reactions.

The invention proposes magnesium lactate (0.2-0.8%) and glycol salicylate (0.5-2.0%) to be used as inhibitors of the histidine decarboxylation. Glycol salicylate is also known in the literature as dermal “counterirritant and anti-inflammatory”.

The antihistaminic action of such molecules was proved experimentally to be synergical with the above-mentioned antioxidants and anti-inflammatory agents such as α-bisabolol (0.2-0.8%) incorporated in its natural matrix, i.e. the extract of chamomile (0.1-0.3%), glycyrrhetinic acid or its esters (0.2-1.0%), rutin (0.05-0.3%), the extract of ginkgo biloba (0.05-0.3%), and aloe gel (0.1-0.6%) which is rich in polysaccharides.

A further characteristic of the invention is to provide in some case the use of benzophenone-3, a known solar filter, whose anti-UV-A and anti-UV-B activity is associated with the anti-UV-2 activity of glycol salicylate.

According to such teachings, some preferred formulations of nutrient anti-wrinkle oils and creams, beauty creams, anti-cellulitis creams, and cleansing milks within the scope of the present invention will be given herebelow only by way of a not limiting example.

Both the preferred proportion and the proportion range allowing the purposes of the present invention to be reached are mentioned for each component in the formulations of the following examples.

### EXAMPLE 1

NUTRIENT ANTI-WRINKLE OILS

| Ingredients (g, ml) | % | range % |
|---|---|---|
| ACTIVE PRINCIPLES | | |
| Sebum-similar oil (A*) | 95 | 93-97 |
| Ubiquinone | 0.1 | 0.02-0.2 |
| Natural vitamin E acetate | 1.0 | 0.5-2.0 |
| Ascorbyl palmitate | 0.2 | |
| α-Bisabolol | 0.2 | 0.1-0.5 |
| Oily extract of chamomile | 0.9 | 0.5-3 |
| EXCIPIENT | | |
| Soya phospholipids | 2.3 | 1.5-4 |
| Sepicide HB | 0.4 | 0.4 |
| SCENT | | |
| Light perfume or others | 0.1 | 0.05-0.3 |

### EXAMPLE 2

NATURAL NUTRIENT SEBUM-SIMILAR CREAM

| Ingredients (g, ml) | % | range % |
|---|---|---|
| ACTIVE PRINCIPLES | | |
| Sebum-similar oil (A*) | 35 | 30-40 |
| Ubiquinone | 0.1 | 0.02-0.2 |
| Natural vitamin E acetate | 1.0 | 0.5-1.5 |
| Glycol salicylate | 1.0 | 0.5-1.5 |
| α-Bisabolol | 0.3 | 0.2-0.6 |
| Extract of chamomile | 0.1 | 0.1-0.3 |
| Glycyrrhetinic acid or its esters | 0.4 | 0.2-1 |
| EXCIPIENT | | |
| Soya phospholipids | 2.0 | 1.0-4 |
| Glycerol | 2.0 | 1.0-4 |
| BHT (butylhydroxytoluene) | 0.1 | |
| Tegin M (glycerol monostearate) | 2.5 | |
| Tegocare 150 | 10.0* | |
| Sepicide HB | 0.4 | |
| Imidazolynidyl urea (IMU) | 0.2 | |
| Bisodium EDTA | 0.1 | |

| | |
|---|---|
| Water | q.s. to 100 |

| | | |
|---|---|---|
| Scent | 0.1 | 0.05-0.3 |

It should be noted that Tegin M, Tegocare 150 and Sepicide HB can be replaced with Microcombin (0.5%), Tegin M (3.8%), Brij 721 (2.0%)., Brij 58 (3.0%), and stearic alcohol (2.5%).

### EXAMPLE 3

IMMUGEN CREAM

| Ingredients (g, ml) | % | range % |
|---|---|---|
| ACTIVE PRINCIPLES | | |
| Sebum-similar oil (A*) | 10 | 6-12 |
| Ubiquinone | 0.05 | 0.05-0.15 |
| Vitamin E acetate | 1.0 | 0.5-1.5 |
| Ascorbyl palmitate | 1.5 | 1-3 |
| α-Bisabolol | 0.3 | 0.2-0.5 |
| Extract of chamomile | 0.1 | 0.1-0.3 |
| Glycol salicylate | 1.0 | 0.5-2 |
| Magnesium lactate | 0.3 | 0.2-0.4 |
| 1-metionine | 0.02 | 0.01-0.03 |
| Benzophenone-3 | 2.0 | 1-3 |
| EXCIPIENT | | |
| Silicone oil 350 | 1.0 | |
| Stearyl alcohol | 1.0 | |
| Xanthan gum | 0.2 | |
| Simulsol 165 V | 1.0 | |
| BHT | 0.1 | |
| Bisodium EDTA | 0.2 | |
| Sepicide HB | 0.4 | |
| Imidazolynidyl urea (IMU) | 0.2 | |
| Brij 721 | 2.0 | |
| Brij 72 | 3.0 | |

| | |
|---|---|
| Water | q.s. to 100 |

| | | |
|---|---|---|
| Scent | 0.05 | 0.005-0.3 |

Carbopol (0.3-0.4%) neutralized with NaOH (0.13-0.15%) can also be used as thickener. In any case, the excipients may vary.
pH 6: adjusting with EDTA or lactic acid
Papaya extract (for foreign creams) 1-2%

BEAUTY CREAMS

| | | | EXAMPLE 6 |
|---|---|---|---|
| | | EXAMPLE 5 | ANTI- |
| | | ANTIAGEING | WRINKLE |
| | EXAMPLE 4 | (dermal | (dermal |
| INGREDIENTS (g, | NUTRIENT | ageing | ageing |
| ml) and range | (young skin) | prevention) | treatment) |
| Active principles [%] | range [%] | range [%] | range [%] |
| Sebum-similar oil | 5 (3 ÷ 6) | 10 (8 ÷ 13) | 15 (13 ÷ 20) |
| (A*) | | | |
| Ubiquinone (0.02 ÷ | 0.05 | 0.05 | 0.05 |
| 0.15) | | | |
| (Natural) vitamin E | 0.5 (0.3 ÷ 0.7) | 0.75 (0.5 ÷ 1) | 1.0 (0.7 ÷ 1.5) |
| acetate | | | |
| Glycol salicylate | 0.5 | 0.5 | 0.5 |
| (0.3 ÷ 1) | | | |
| Ascorbyl dipalmi- | 1.0 | 1.2 | 1.5 |
| tate (1 ÷ 3) | | | |
| Magnesium lactate | 0.3 | 0.3 | 0.3 |
| (0.2 ÷ 0.7) | | | |
| α-Bisabolol (0.2 ÷ | 0.3 | 0.3 | 0.3 |
| 0.6) | | | |
| Extract of chamo- | 0.1 | 0.1 | 0.1 |
| mile (0.1 ÷ 0.3) | | | |
| Glycyrrhetinic acid | 0.5 | 0.5 | 0.5 |
| (0.2 ÷ 0.8) (or its | | | |
| esters) | | | |
| Rutin (0.05 ÷ 0.15) | 0.05 | 0.05 | 0.05 |
| Extract of ginkgo | 0.1 | 0.2 | 0.3 |
| biloba (0.1 ÷ 0.5) | | | |
| Collagen hydrolisate | 0.5 | 0.5 | 0.5 |
| (0.2 ÷ 0.8) | | | |
| Aloe gel (200:1) | 0.1 | 0.1 | 0.1 |
| (0.1 ÷ 0.5) | | | |
| Hyaluronic acid | 0.1 | 0.1 | 0.1 |
| (0.05 ÷ 0.2) | | | |
| * | | | |

| Excipients: [%] | [%] | [%] | [%] |
|---|---|---|---|
| Glycerol | 3.0 | 2.0 | 2.0 |
| Cetostearyl alcohol | 1 | 1.2 | 1.5 |
| Brij 721 | 2.0 | 2.0 | 2.0 |
| Brij 72 | 3.0 | 3.0 | 3.0 |
| Tween 60 | 2.0 | 2.0 | 2.0 |
| BHT | 0.1 | 0.1 | 0.1 |
| Silicone oil 350 | 0.5 | 0.5 | 0.5 |
| Sepicide HB | 0.4 | 0.4 | 0.4 |
| Imidazolynidyl urea | 0.2 | 0.2 | 0.2 |
| EDTA | 0.1 | 0.1 | 0.1 |
| Light perfume | 0.05 | 0.05 | 0.05 |
| (0.05 ÷ 0.3) | | | |

Carbopol (0.3 ÷ 0.4%) neutralized with NaOH (0.13 ÷ 0.15%) can also be used as thickener. In any case the excipients may vary.
pH 6: adjusting with EDTA or lactic acid.
(*) Papaya extract (for foreign creams) 1-2%

CLEANSING MILK

| | | |
|---|---|---|
| | Example 7 | Example 8 |
| INGREDIENTS (g, ml) and | Normal dry | Normal fat |
| range | skin | skin |

| | | |
|---|---|---|
| Active principles [%] | [%] | [%] |

| Sebum-similar oil (A*) (1-5) | 3 | 1.5 |
|---|---|---|
| Natural vitamin E acetate (0.1.- | 0.3 | 0.3 |
| 0.5) | | |
| Magnesium lactate (0.1 ÷ 0.5) | 0.3 | 0.3 |
| Glycol salicylate (0.3 ÷ 1) | 0.5 | 0.5 |
| α-Bisabolol (0.1 ÷ 0.8) | 0.4 | 0.4 |
| Extract of chamomile (0.1 ÷ 0.3) | 0.1 | 0.1 |
| Glycyrrhetinic acid (0.1 ÷ 0.6) | 0.2 | 0.2 |
| (or its esters) | | |
| Aloe gel (200:1) (0.1 ÷ 0.5) | 0.3 | 0.2 |
| Extract of ginkgo biloba | 0.1 | 0.1 |
| (0.02 ÷ 0.15) | | |
| Mucopolysaccharides (0.1 ÷ 0.5) | 0.1 | 0.1 |
| Excipients: [%] | [%] | [%] |
| Glycerol | 2.0 | 2.0 |
| Soya lecithin (granules) (1-5) | 2.0 | 2.0 |
| Cetylic alcohol | 0.5 | 0.5 |
| Cetostearyl alcohol | 1.4 | 1.4 |
| Brij 721 | 3.0 | 3.0 |
| Brij 72 | 0.5 | 0.5 |
| BHT | 0.1 | 0.1 |
| Silicone oil 350 | 0.5 | 0.5 |
| Sepicide HB | 0.3 | 0.3 |
| Imidazolynidyl urea | 0.2 | 0.2 |
| Bisodium EDTA | 0.1 | 0.1 |
| Light perfume (0.05 ÷ 0.3) | 0.05 | 0.05 |
| H₂O | q.s.to 100 | q.s. to 100 |

Sodium carbonate or NaOH to increase pH to 6
Papaya extract (for foreign creams) 1%

MOISTURIZING CREAM

| | EXAMPLE 9 | EXAMPLE 10 |
|---|---|---|
| INGREDIENTS (g, ml) and | Normal dry | Normal fat |
| range | skin | skin |
| Active principles [%] | [%] | [%] |
| Sebum-similar oil (A*) (1-5) | 4 | 2 |
| Natural vitamin E acetate (0.2- | 0.3 | 0.3 |
| 0.5) | | |
| α-Bisabolol (0.1 ÷ 0.5) | 0.4 | 0.4 |
| Extract of chamomile (0.1 ÷ 0.3) | 0.1 | 0.1 |
| Glycyrrhetinic acid (0.1 ÷ 0.6) | 0.3 | 0.3 |
| (or its esters) | | |
| Aloe gel (200:1) (0.1 ÷ 0.5) | 0.1 | 0.1 |
| Extract of ginkgo biloba | 0.1 | 0.1 |
| (0.1 ÷ 0.3) | | |
| Soya phospholipids (0.5 ÷ 3) | 1.0 | 1.0 |
| Glycol salicylate (0.2 ÷ 0.5) | 0.4 | 0.4 |
| Ubiquinone (0.02 ÷ 0.05) | 0.03 | 0.03 |
| Dry extract of rutin (0.05 ÷ 0.2) | 0.1 | 0.1 |
| Proteic collagen hydrolisate | 0.3 | 0.3 |
| (0.1 ÷ 0.5) | | |
| Ascorbyl dipalmitate (0.5 ÷ 3) | 0.6 | 0.6 |
| Excipients: [%] | [%] | [%] |
| Glycerol | 3.0 | 3.0 |
| Cetylic alcohol | 0.5 | 0.5 |
| Tween 60 | 2.0 | 2.0 |
| Brij 721 | 2.0 | 2.0 |
| Brij 72 | 3.0 | 3.0 |
| BHT | 0.1 | 0.1 |
| Silicone oil 350 | 0.5 | 0.5 |
| Sepicide HB | 0.3 | 0.3 |
| Imidazolynidyl urea | 0.2 | 0.2 |
| Bisodium EDTA | 0.1 | 0.1 |
| Light perfume (0.05 ÷ 0.3) | 0.05 | 0.05 |
| H₂O | q.s. to 100 | q.s. to 100 |

Carbopol (0.3 ÷ 0.4%) neutralized with NaOH (0.13 ÷ 0.15%) can also be used as thickener. In any case the excipients may vary.
pH 6: adjusting with EDTA or lactic acid.
Papaya extract (for foreign creams) 1-2%

It is self-evident from the examples disclosed above that a number of modifications and changes may be made to the formulations as far as the active principles is concerned without departing from the scope of the invention. Therefore, such modifications and changes, including the variations of nature and composition of the excipients, fall within the scope of the following claims which are intended to cover the general, specific features of the invention.

1. A cosmetic formulation for topical use, including a mixture of active principles of natural origin fulfilling the following requirements:
having a lipid composition close to the human sebum; having a pool of antioxidants similar to those present in sebum and skin;
having anti-inflammatory and anti-free-radical molecules also capable of preventing histidine from being decarboxylated and then histamine to be formed.

2. The cosmetic formulation of claim 1, including: a mixture of macadamia oil and avocado oil in the same proportion as triglycerides in sebum; unsaponifiable olive oil in a proportion corresponding to squalene in sebum; jojoba wax in a proportion corresponding to waxes in sebum; free, esterified cholesterol in the same quantities as those in human sebum; a quantity of vitamin E (s-RRR-α-tocopherol) and ubiquinone as physiological antioxidants to protect sebum from oxidation induced by solar radiation and other oxidizing agents in the environment; an effective quantity of magnesium lactate and glycol salicylate as inhibitor of the histidine decarboxylation.

3. The cosmetic formulation of the preceding claims, characterized in that vitamin E and ubiquinone are used in association with effective quantities of ascorbyl dipalmitate, a steady form of vitamin C, or magnesium ascorbyl phosphate.

4. The cosmetic formulation of the preceding claims, further including an effective quantity of active flavonoids such as rutin and ginkgoales of ginkgo biloba as antioxidants.

5. The cosmetic formulation of the preceding claims, further including an effective quantity of α-bisabolol in its natural matrix and glycyrrhetinic acid or its esters to provide a synergic antihistaminic effect of magnesium lactate and glycol salicylate.

6. The cosmetic formulation of the preceding claims, further including benzophenone-3, whose anti-LTV-A and anti-UV-B activity is associated with the anti-UV-B activity of glycol salicylate.

7. A nutrient, anti-wrinkle oil for topical use on skin, including the following active principles in weight percentages of the total weight of the product:

| | |
|---|---|
| Sebum-similar oil (A*) of the known type | 93-97% |
| Ubiquinone | 0.02-0.2% |
| Natural vitamin E acetate | 0.5-2.0% |
| Ascorbyl palmitate | about 0.2% |
| α-Bisabolol | 0.1-0.5% |
| Oily extract of chamomile | 0.5-3% |

8. A natural nutrient, sebum-similar cream for topical use on skin, including the following active-principles in weight percentages of the total weight of the product:

| | |
|---|---|
| Sebum-similar oil (A*) of the known type | 30-40% |
| Ubiquinone | 0.02-0.2% |
| Natural vitamin E acetate | 0.5-1.5% |
| Glycol salicylate | 0.5-1.5% |
| α-Bisabolol | 0.2-0.6% |
| Extract of chamomile | 0.1-0.3% |
| Glycyrrhetinic acid or its esters | 0.2-1% |

9. A protective cream for topical use on skin, including the following active principles in weight percentages of the total weight of the composition:

| | |
|---|---|
| Sebum-similar oil (A*) of the known type | 6-12 |
| Ubiquinone | 0.05-0.15 |
| Vitamin E acetate | 0.5-1.5 |
| Ascorbyl palmitate | 1-3 |
| Glycol salicylate | 0.5-2 |
| Magnesium lactate | 0.2-0.4 |
| α-Bisabolol | 0.2-0.5 |
| Extract of chamomile | 0.1-0.3 |
| 1-metionine | 0.01-0.03 |
| Benzophenone-3 | 1-3 |

10. Nutrient, beauty cream for topical use on young skins, including the following active principles in weight percentage of the total weight of the composition:

| | % |
|---|---|
| Sebum-similar oil (A*) | 3 ÷ 6 |
| Ubiquinone | 0.02 ÷ 0.15 |
| (Natural) vitamin E acetate | 0.3 ÷ 0.7 |
| Glycol salicylate | 0.3 ÷ 1 |
| Ascorbyl dipalmitate | 1 ÷ 3 |
| Magnesium lactate | 0.2 ÷ 0.7 |
| α-Bisabolol | 0.2 ÷ 0.6 |
| Extract of chamomile | 0.1 ÷ 0.3 |
| Glycyrrhetinic acid (or its | 0.2 ÷ 0.8 |
| esters) | |
| Rutin | 0.05 ÷ 0.15 |
| Extract of ginkgo biloba | 0.1 ÷ 0.5 |
| Collagen hydrolisate | 0.5 |
| Aloe gel (200:1) | 0.1 ÷ 0.5 |
| Hyaluronic acid | 0.05 ÷ 0.2 |

11. A beauty cream for topical use for the prevention of skin ageing, including the following active principles in weight percentage of the total weight of the composition:

| | % |
|---|---|
| Sebum-similar oil (A*) | 8 ÷ 13 |
| Ubiquinone | 0.02 ÷ 0.15 |
| (Natural) vitamin E acetate | 0.5 ÷ 1 |
| Glycol salicylate | 0.3 ÷ 1 |
| Ascorbyl dipalmitate | 1 ÷ 3 |
| Magnesium lactate | 0.2 ÷ 0.7 |
| α-Bisabolol | 0.2 ÷ 0.6 |
| Extract of chamomile | 0.1 ÷ 0.3 |
| Glycyrrhetinic acid (or its | 0.2 ÷ 0.8 |
| esters) | |
| Rutin | 0.05 ÷ 0.15 |
| Extract of ginkgo biloba | 0.1 ÷ 0.5 |
| Collagen hydrolisate | 0.2 ÷ 0.8 |
| Aloe gel (200:1) | 0.1 ÷ 0.5 |
| Hyaluronic acid | 0.05 ÷ 0.2 |
| Papaya extract (for foreign | 1 ÷ 2 |
| creams) | |

12. An anti-wrinkle beauty cream for the topical treatment of the skin ageing, including the following active principles in weight percentage of the total weight of the composition:

| | % |
|---|---|
| Sebum-similar oil (A*) | 13 ÷ 20 |
| Ubiquinone | 0.02 ÷ 0.15 |
| (Natural) vitamin E acetate | 0.7 ÷ 1.5 |
| Glycol salicylate | 0.3 ÷ 1 |
| Ascorbyl dipalmitate | 1 ÷ 3 |
| Magnesium lactate | 0.2 ÷ 0.7 |
| α-Bisabolol | 0.2 ÷ 0.6 |
| Extract of chamomile | 0.1 ÷ 0.3 |
| Glycyrrhetinic acid (or its | 0.2 ÷ 0.8 |
| esters) | |
| Rutin | 0.05 ÷ 0.15 |
| Extract of ginkgo biloba | 0.1 ÷ 0.5 |
| Collagen hydrolisate | 0.2 ÷ 0.8 |
| Aloe gel (200:1) | 0.1 ÷ 0.5 |
| Hyaluronic acid | 0.05 ÷ 0.2 |
| Papaya extract (for foreign | 1 ÷ 2 |
| creams) | |

13. A cleansing milk for topical use on skin, including the following active principles in weight percentage of the total weight of the composition:

| | % |
|---|---|
| Sebum-similar oil (A*) | 1 ÷ 5 |
| Natural vitamin E acetate | 0.1 ÷ 0.5 |
| Magnesium lactate | 0.1 ÷ 0.5 |
| Glycol salicylate | 0.3 ÷ 1 |
| α-Bisabolol | 0.1 ÷ 0.8 |
| Extract of chamomile | 0.1 ÷ 0.3 |
| Glycyrrhetinic acid (or its | 0.1 ÷ 0.6 |
| esters | |
| Aloe gel (200:1) | 0.1 ÷ 0.5 |
| Dry extract of ginkgo biloba | 0.05 ÷ 0.2 |
| Mucopolysaccharides | 0.1 ÷ 0.5 |
| Papaya extract for foreign | 1 |
| creams) | |

14. A hydrating milk for topical use on skin, including the following active principles in weight percentage of the total weight of the composition:

| | % |
|---|---|
| Sebum-similar oil (A*) | 1 ÷ 5 |
| Natural vitamin E acetate | 0.2 ÷ 0.5 |
| α-Bisabolol | 0.1 ÷ 0.5 |
| Extract of chamomile | 0.1 ÷ 0.3 |
| Glycyrrhetinic acid (or its | 0.1 ÷ 0.6 |
| esters) | |
| Aloe gel (200:1) | 0.1 ÷ 0.5 |
| Extract of ginkgo biloba | 0.1 ÷ 0.3 |
| Soya phospholipids | 0.5 ÷ 3 |
| Glycol salicylate | 0.2 ÷ 0.5 |
| Ubiquinone | 0.02 ÷ 0.05 |
| Dry extract of rutin | 0.05 ÷ 0.2 |
| Proteic hydrolisate collagen | 0.1 ÷ 0.5 |
| Ascorbyl dipalmitate | 0.5 ÷ 3 |
